(19) United States
(12) Patent Application Publication
(10) Pub. No.: US 2004/0266713 A1
(43) Pub. Date: Dec. 30, 2004

(54) Methods and compositions for solid tumor treatment

(76) Inventors: Shan Lu, Franklin, MA (US); Te-Hui W. Chou, Wayland, MA (US); Shixia Wang, Northborough, MA (US)

Correspondence Address: FISH & RICHARDSON PC, 225 FRANKLIN ST, BOSTON, MA, 02110, US

(21) Appl. No.: 10/830,810

(22) Filed: Apr. 23, 2004

Related U.S. Application Data
(60) Provisional application No. 60/464,700, filed on Apr. 23, 2003.

(51) Int. Cl. A61K 48/00; C07H 21/04; C07K 14/535; C07K 14/54

(52) U.S. Cl. 514/44; 536/23.5; 435/69.5; 435/320.1; 435/325; 530/351

(57) ABSTRACT

Nucleic acid compositions and methods of use in treating solid tumors are disclosed. The nucleic acid compositions include a plurality of nucleic acid plasmids. Each plasmid encodes a single protein, either a cytokine or an anti-tumor protein. Each composition includes a plurality of these nucleic acid plasmids that encode a combination of two or more cytokines, a combination of cytokines and anti-tumor proteins, a combination of anti-tumor proteins, or an anti-tumor protein. The nucleic acid compositions of the invention can be delivered locally to a solid tumor.

### CROSS-REFERENCE TO RELATED APPLICATIONS

This application claims the benefit of priority of U.S. Ser. No. 60/464,700, filed Apr. 23, 2003, the contents of which are hereby incorporated by reference in their entirety.

### TECHNICAL FIELD

This invention relates to compositions and methods for the treatment of cancer.

### BACKGROUND

Malignant solid tumors have been commonly treated by surgery, chemotherapy, and radiation. These treatment approaches are sometimes only palliative. Recent efforts to treat malignant solid tumors have applied immunomodulatory strategies, including administration of genetically modified tumor cells, dendritic cells either pulsed or transduced with tumor-associated antigens, immunization with soluble proteins or synthetic peptides, recombinant viruses or bacteria encoding tumor-associated antigens, and naked plasmid DNA encoding tumor-associated antigens (Haupt et al., 2002, Exp Biol Med 227:227-237).

The goal in using immunomodulatory strategies, such as anti-tumor vaccines, is to induce specific immunological responses to tumor-associated antigens, e.g., by triggering destruction of tumor cells, and to protect patients from relapses (e.g., via cell-mediated immune responses, e.g., cytotoxic T cell (CTL) responses, and via humoral immune responses, e.g., antibody-mediated cytotoxicity). Thus, a persistent anti-tumor immune memory response can be established by the induction of expanded T and B lymphocytes, which first recognize and then react against tumor-associated antigens with specificity and highly destructive potential (Haupt et al., 2002, Exp Biol Med 227:227-237). Many immunomodulatory strategies for tumor treatment have relied on systemic administration of immune-stimulatory agents, such as cytokines. Side effects from systemically delivered immunomodulatory treatment remains a concern since large doses are often required for efficacy.

A recently developed approach to induce cell-mediated, humoral immune responses, and other cytotoxic effects directed against tumors is the DNA vaccine. At the cellular level, upon administration of a plasmid DNA vaccine expressing a selected tumor antigen, the plasmid DNA enters a host cell nucleus where the plasmid can persist, e.g., as a circular non-replicating episome. The persistence of the plasmid, which is not integrated into the genome of the host, results in long-term expression of the plasmid DNA-encoded protein(s) by the host cell's machinery. The expressed antigen, encoded by the plasmid DNA is then processed through what is referred to as the endogenous and exogenous pathways. These pathways allow the presentation of peptide antigen by both major histocompatibility complexes (MHC) class I and class II, leading to the stimulation of both cell-mediated and humoral responses (Wolff et al., 1990, Science 247:1465-1468). DNA vaccines can also be used to express toxic proteins within tumor cells, thereby leading to direct destruction of the cells and minimizing damage to non-cancerous tissue.

### SUMMARY

The invention is based, in part, on the discovery that intratumoral administration of naked DNA molecules that encode cytokines and non-cytokine anti-tumor proteins can inhibit tumor growth in vivo. Accordingly, the invention features nucleic acid compositions (e.g., naked DNA plasmids) that are administered by direct intratumoral injection or other modes of localized administration and that, as a group, express one or a combination of proteins useful in treating solid tumors (e.g., useful in preventing the progression and/or growth of benign tumors into cancerous tumors, e.g., useful in stimulating destruction of tumor cells by immune or non-immune mechanisms). The nucleic acid compositions can express a combination of two or more different cytokines (e.g., interleukin-2 (IL-2) and interleukin-10 (IL-10)); a combination of one or more cytokines with one or more non-cytokine anti-tumor proteins (e.g., IL-2, IL-10, and tumor necrosis factor (TNF)-related apoptosis-inducing ligand (TRAIL)); a single non-cytokine anti-tumor protein (e.g., TRAIL or a truncated version of TRAIL); or a combination of more than one non-cytokine anti-tumor protein when injected into a benign or malignant tumor (e.g.,TRAIL and phosphatase and tensin homolog (PTEN)). Each combination (described, supra) can include a plurality of nucleic acids with each type of nucleic acid encoding a single cytokine or anti-tumor protein.

In one aspect, the invention features isolated nucleic acid compositions that include a plurality of sets of nucleic acid molecules (e.g., DNA vectors). The plurality of sets of nucleic acid molecules include, e.g., a first set of nucleic acid molecules and a second set of nucleic acid molecules wherein (a) each nucleic acid molecule of the first set of the plurality includes a sequence encoding a first cytokine or functional fragment thereof and each nucleic acid molecule of the second set of the plurality includes a sequence encoding a second cytokine or functional fragment thereof, (b) each nucleic acid molecule of the first set of the plurality includes a sequence encoding a first cytokine or functional fragment thereof and each nucleic acid molecule of the second set of the plurality includes a sequence encoding a first non-cytokine anti-tumor protein or functional fragment thereof, or (c) each nucleic acid molecule of a first set of the plurality includes a sequence encoding a first non-cytokine anti-tumor protein or functional fragment thereof and each nucleic acid molecule of the second set of the plurality includes a sequence encoding a second non-cytokine anti-tumor protein or functional fragment thereof.

The cytokines can be selected from the group consisting of IL-2, IL-4, IL-5, IL-10, IL-12, IL-15, IL-18, GM-CSF, TGF-β, TNF-α, and IFN-γ. The cytokines can be mammalian cytokines, e.g., human cytokines.

The non-cytokine anti-tumor proteins can be selected from the group consisting of an apoptotic protein, an anti-angiogenic protein, and a cell cycle arrest protein. For example, the non-cytokine anti-tumor proteins of (b) or (c) can be selected from the group consisting of tumor necrosis factor (TNF)-related apoptosis-inducing ligand (TRAIL), phosphatase and tensin homolog (PTEN), suppressor of high-copy phosphoprotein phosphatase 1 (SHP1), and SHP2. The non-cytokine anti-tumor proteins of (b) or (c) can be human non-cytokine anti-tumor proteins.

The nucleic acid composition can include a first set of nucleic acid molecules and a second set of nucleic acid molecules wherein each nucleic acid molecule of the first set of the plurality includes a sequence encoding a first cytokine or functional fragment thereof and each nucleic acid molecule of the second set of the plurality includes a sequence encoding a second cytokine or functional fragment thereof. In one embodiment, each nucleic acid molecule of the first set of includes a sequence encoding IL-2 or a functional fragment thereof, e.g., a sequence encoding IL-2 that is at least 80% 80% (e.g., at least 80%, 85%, 90%, 95%, 97%, 99%, or 100%) identical to SEQ ID NO:1, and, optionally, each nucleic acid molecule of the second set includes a sequence encoding IL-10 or a functional fragment thereof. In one embodiment, each nucleic acid molecule of the first set of includes a sequence encoding IL-10 or a functional fragment thereof, e.g., a sequence at least 80% 80% (e.g., at least 80%, 85%, 90%, 95%, 97%, 99%, or 100%) identical to SEQ ID NO:2.

The nucleic acid composition can further include a third set of nucleic acid molecules, wherein each nucleic acid molecule of the third set includes a sequence encoding a non-cytokine anti-tumor protein or functional fragment thereof, e.g., TRAIL or a functional fragment thereof, e.g., a sequence at least 80% (e.g., at least 80%, 85%, 90%, 95%, 97%, 99%, or 100%) identical to SEQ ID NO:3. The non-cytokine anti-tumor protein can be PTEN or a functional fragment thereof and the sequence encoding PTEN is, e.g., at least 80% (e.g., at least 80%, 85%, 90%, 95%, 97%, 99%, or 100%) identical to SEQ ID NO:4.

The nucleic acid composition can include a first set of nucleic acid molecules and a second set of nucleic acid molecules wherein each nucleic acid molecule of the first set of the plurality includes a sequence encoding a first cytokine or functional fragment thereof and each nucleic acid molecule of the second set of the plurality includes a sequence encoding a first non-cytokine anti-tumor protein or functional fragment thereof.

In one embodiment, each nucleic acid molecule of the first set includes a sequence encoding IL-2 or a functional fragment thereof, e.g., a sequence at least 80% (e.g., at least 80%, 85%, 90%, 95%, 97%, 99%, or 100%) identical to SEQ ID NO:1. In one embodiment, each nucleic acid molecule of the first set includes a sequence encoding IL-10 or a functional fragment thereof, e.g., a sequence at least 80% (e.g., at least 80%, 85%, 90%, 95%, 97%, 99%, or 100%)identical to SEQ ID NO:2.

The non-cytokine anti-tumor protein can be TRAIL or a functional fragment thereof (e.g., a fragment lacking a transmembrane domain of TRAIL). The sequence encoding TRAIL can be at least 80% (e.g., at least 80%, 85%, 90%, 95%, 97%, 99%, or 100%) identical to SEQ ID NO:3. The non-cytokine anti-tumor protein can be PTEN or a functional fragment thereof. The sequence encoding PTEN can be at least 80% (e.g., at least 80%, 85%, 90%, 95%, 97%, 99%, or 100%) identical to SEQ ID NO:4.

The nucleic acid composition can include a first set of nucleic acid molecules and a second set of nucleic acid molecules wherein each nucleic acid molecule of a first set of the plurality includes a sequence encoding a first non-cytokine anti-tumor protein or functional fragment thereof and each nucleic acid molecule of the second set of the plurality includes a sequence encoding a second non-cytokine anti-tumor protein or functional fragment thereof.

In one embodiment, the first non-cytokine anti-tumor protein is TRAIL or a functional fragment thereof and, e.g., the sequence encoding TRAIL is at least 80% (e.g., at least 80%, 85%, 90%, 95%, 97%, 99%, or 100%) identical to SEQ ID NO:3. The nucleic acid composition can further include a third set of nucleic acid molecules, each nucleic acid molecule of the third set including a sequence encoding a cytokine or functional fragment thereof The invention also features a nucleic acid composition including a first set of nucleic acid vectors, each vector of the first set including a sequence encoding IL-2 or a functional fragment thereof, a second set of nucleic acid vectors, each vector of the second set including a sequence encoding IL-10 or a functional fragment thereof, and a third set of nucleic acid vectors, each vector of the third set including a sequence encoding TNF-related apoptosis-inducing ligand (TRAIL) or a functional fragment thereof.

In another aspect, the invention features pharmaceutical composition consisting essentially of the nucleic acid composition described herein (e.g., a nucleic acid composition comprising one or more sets of nucleic acid molecules comprising a sequence encoding a cytokine and one or more sets of nucleic acid molecules comprising a sequence encoding an anti-tumor protein) and a pharmaceutically acceptable carrier. The pharmaceutically acceptable carrier can include saline or buffered saline, but may lack any adjuvants or facilitating agents.

The invention also features kits that include a nucleic acid composition described herein, and instructions for intratumoral administration of the composition, e.g., without an adjuvant or facilitating agent.

In another aspect, the invention features methods of treating a solid tumor in a subject. The methods include locally administering a pharmaceutical composition described herein to the solid tumor of the subject. The composition can be administered by intratumoral injection. The composition can be administered by subcutaneous injection, intramuscular injection, endoscopic percutaneous injection, injection into a vessel supplying blood flow to the solid tumor, injection into a vessel supplying blood flow to a tumor-containing organ, inhalation, localized topical administration, mucosal administration, injection into cerebral spinal fluid, by osmotic pump, or by lymphoid injecting into circulation. In these methods, the pharmaceutical composition can lack an adjuvant or facilitating agent, and the tumor can be a prostate tumor or a head and neck tumor.

The compositions can be administered in combination with a second agent or treatment modality, e.g., a chemotherapeutic agent, radiation, or surgery.

The invention also includes a method for simultaneously eliciting an immune response against cancerous cells and decreasing or blocking cancer cell proliferation in a patient in need thereof. The method includes, for example, administering to the patient a composition including a first set of nucleic acid molecules and a second set of nucleic acid molecules wherein each nucleic acid molecule of the first set of the plurality includes a sequence encoding a first cytokine (e.g. ,IL-2 or IL-10) or functional fragment thereof and each nucleic acid molecule of the second set of the plurality includes a sequence encoding a first non-cytokine anti-tumor protein or functional fragment thereof (e.g., TRAIL). The composition can be administered without an adjuvant or facilitating agent.

“Anti-tumor proteins” are a number of different non-cytokine proteins which have been shown to inhibit the growth or proliferation of a cancerous cell. As used herein, an anti-tumor protein can be a non-cytokine apoptotic protein (e.g., TRAIL), cell cycle arrest protein, or anti-angiogenic protein.

“Localized delivery or administration” refers to delivery other than systemic delivery of the compositions described herein. Localized delivery can be delivery on, or near, or into a tumor. Localized delivery includes “intratumoral delivery,” in which a composition is injected directly into a tumor. Localized delivery also includes the administration of a composition into a vessel supplying an organ containing a tumor (e.g., administration into the portal vein to treat a hepatoma). Another example of localized delivery includes inhalation of a composition for localized delivery to the lungs (e.g., as may be needed in widespread micrometastases in the lung).

The compositions described herein lack a “facilitating agent” or “adjuvant.” This means they are in a solution of saline or buffered saline and do not rely on a facilitating agent, which is an agent that aids the entry of molecules into target cells (e.g., tumor cells) or an adjuvant, which aids molecules to elicit an immune response. As used herein, the addition of cytokines to the combination delivered does not serve as the addition of an adjuvant. Typical adjuvants include water emulsions (e.g., complete and incomplete Freund's adjuvant), oil, Corynebacterium parvum, Bacillus Calmette Guerin, iron oxide, sodium alginate, aluminum hydroxide, aluminum and calcium salts (i.e., alum), unmethylated CpG motifs, glucan, and dextran sulfate. Typical facilitating agents include lipid carriers, liposomes, proteoliposomes or buprokane. None of these are required in the methods and compositions of the invention.

The nucleic acid constructs can encode full-length proteins (e.g., cytokines and non-cytokine anti-tumor proteins) as well as truncated, “functional” fragments. A functional fragment is a portion of the protein that is capable of performing a biological function of interest. For example, as seen in Example 1, infra, a truncated TRAIL which starts at amino acid 93 is more readily expressed in cell lines and is able to induce apoptosis (see FIG. 4). Thus, TRAIL 47 and TRAIL 93 are both functional fragments of the TRAIL protein. A functional fragment of a cytokine is a fragment that mediates the signaling function of the cytokine which promotes an immune response to the tumor. One of ordinary skill in the art would be able to suitably test for a specific function (e.g., induction of apoptosis, signaling through a cytokine receptor) when provided a fragment of a protein of interest, and thus could readily determine if a fragment is a functional fragment.

As used herein, the term “substantially identical” refers to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have at least 80% sequence identity.

Calculations of “identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 50% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. The percent identity between two sequences is determined using the Needleman and Wunsch, J. Mol. Biol., 48:444-453, 1970, algorithm which has been incorporated into the GAP program in the GCG software package, using a Blossum 62 scoring matrix with a gap penalty of 12, a gap extend penalty of 4, and a frameshift gap penalty of 5. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a representation of a western blot showing expression of PTEN from the pJW4303 vector in 293T cells. (S: supernatant, L: lysate; lanes 1 and 2 contain samples from cells transfected with a vector expressing PTEN, lanes 3 and 4 contain samples from cells transfected with empty vector). The arrow designates the band representing PTEN protein. FIG 1B is a representation of a western blot showing expression of TRAIL from the pJW4303 vector in 293T cells. (S: supernatant, L:lysate; lanes 1 and 2 contain samples from cells transfected with a vector expressing TRAIL, lanes 3 and 4 contain samples from cells transfected with empty vector ). The arrow designates the band representing TRAIL protein.

FIG. 2 is a representation of a western blot showing expression of TRAIL in LNCAP cells. Lane 1 contains a sample from 293 T cells transfected with an empty pJW4303 vector. Lane 2 contains a sample from 293 T cells transfected with a TRAIL-encoding vector. Lane 3 contains a sample from LnCaP cells transfected with an empty pJW4303 vector. Lane 4 contains a sample from LnCaP cells transfected with a TRAIL-encoding vector. The arrow designates the position of the band representing expressed TRAIL protein. The migration of protein molecular weight markers is indicated on the left side of the blot.

FIG. 3 is a representation of a western blot showing expression of the empty pJW4303 vector (lanes 1 and 2), wild type TRAIL (lanes 3 and 4), a truncated form of TRAIL which starts at amino acid 47 (lanes 5 and 6), and truncated TRAIL which starts at amino acid 93 (lanes 7 and 8). (S: supernatant, L: lysate). The migration of protein molecular weight markers is indicated on the left side of the blot.

FIG. 4 is a bar graph showing the percentage of cells undergoing apoptosis. The first bar represents the percentage of untransfected 293T cells undergoing apoptosis. The second bar represents the percentage of 293T cells, transfected with wild type TRAIL, undergoing apoptosis. The third bar represents the percentage of 293T cells, transfected with truncated TRAIL starting at amino acid 47, undergoing apoptosis. The fourth bar represents the percentage of 293T cells, transfected with truncated TRAIL starting at amino acid 93, undergoing apoptosis. The fifth bar represents the percentage of 293T cells, transfected with pJW4303 vector, undergoing apoptosis.

FIG. 5A and FIG. 5B are representations of tumor tissue after immuno-histochemistry probing with rabbit anti-TRAIL sera. Positive TRAIL expression is seen in darkened regions. FIG. 5A is a photograph of tumor tissue inoculated with pJW4303 vector as negative control. FIG. 5B is a photograph of tumor tissue inoculated with TRAIL.

FIG. 6 is a graph that depicts survival curves of LNCaP tumor bearing mice treated with the designated naked DNA construct composition by direct intratumoral injection. Survival of control animals (animals injected with empty pJW4303 vector), are represented by open squares. Open circles designate the survival curve for mice inoculated with DNA compositions encoding IL-2 and IL-10. Dots designate the survival curve for mice inoculated with DNA encoding TRAIL.

FIG. 7 is a graph that depicts survival curves of mice bearing head and neck-derived tumors treated with the designated naked DNA construct composition by direct intratumoral injection. Open squares designate the survival curve for the control group, which was inoculated with pJW4303 vector. Open circles designate the survival curve for the group inoculated with DNA encoding IL-2 and IL-10. Dots designate the survival curve for mice inoculated with DNA encoding TRAIL.

FIG. 8A is a representation of the nucleic acid sequence of human IL-2 (SEQ ID NO:1).

FIG. 8B is a representation of the nucleic acid sequence of human IL-10 (SEQ ID NO:2).

FIG. 9 is a representation of the nucleic acid sequence of human TRAIL (SEQ ID NO:3).

FIG. 10 is a representation of the nucleic acid sequence of human PTEN (SEQ ID NO:4). Like reference symbols in the various drawings indicate like elements.

### DETAILED DESCRIPTION

It has been discovered that new nucleic acid compositions can be administered intratumorally to express polypeptides that inhibit tumor growth in vivo. In particular, cytokines and/or anti-tumor proteins expressed by intratumorally-administered nucleic acids can be used to treat solid tumors. Thus, the invention provides nucleic acid compositions (e.g., DNA constructs, e.g., naked DNA vectors or plasmids) that include a plurality of nucleic acid constructs. The invention also provides methods for administration of the compositions to subjects (e.g., methods for administering the compositions to a tumor locally). Each one of a plurality of constructs can encode a single protein (e.g., a single therapeutic protein, e.g., a single immunomodulatory protein or a single non-cytokine anti-tumor protein) and the plurality as a whole can encode a single type of polypeptide or a combination of polypeptides. Thus, the plurality of constructs, e.g., plasmids, includes members that each encode the same type of protein or that each encode different proteins. In general, the composition will include two or more sets of plasmids, in which all plasmids within a set encode the same protein.

A plurality of plasmids can encode one or more cytokines or other immunomodulatory molecules that can induce cell-mediated and/or humoral immunity against tumor cells. For example, the invention provides a plurality of plasmids, e.g., a set of nucleic acid plasmids that encode one type of cytokine (e.g., IL-2, IL-4, IL-5, IL-10, IL-12, IL-15, IL-18, GM-CSF, TNF-α, TGF-β, IFN-γ, CD80, CD86, or a functional fragment of any of these) in combination with a set of nucleic acid plasmids that all encode a second, different cytokine. The invention also provides that a plurality of plasmids includes a set of nucleic acid plasmids that all encode the same cytokine combined with a set of nucleic acid plasmids that encode the same anti-tumor protein (e.g., TRAIL, PTEN, Suppressor of High-copy Phosphoprotein phosphatase 1 (SHP1), SHP2, and functional fragments of any of these). The invention also provides that a plurality of plasmids includes a set of nucleic acid plasmids that all encode the same cytokine, a set of nucleic acid plasmids that all encode a second different cytokine, and a set of nucleic acid plasmids that all encode an anti-tumor protein (or functional fragment of an anti-tumor protein). The invention also provides that the plurality of plasmids includes a set of nucleic acid plasmids that all encode a non-cytokine anti-tumor protein, not in combination with a cytokine. The invention also provides that the plurality of plasmids includes a two or more sets of plasmids, each set encoding a different non-cytokine anti-tumor protein. When delivered, such combinations can elicit an immune response simultaneously to eliciting anti-tumor activity. Such a simultaneous response against the cancer cells using different mechanisms has a powerful effect against tumor cell growth and progression.

In one embodiment the naked plasmid DNA composition contains a set of nucleic acid plasmids that encode IL-2 and a set of nucleic acid plasmids that encode IL-10. In other embodiments, the naked DNA plasmids can each encode a functional fragment of a cytokine or of an anti-tumor protein. For example, the naked DNA plasmids can encode a truncated TRAIL protein which starts at amino acid 93 of the protein. In another example, the naked DNA plasmids can include a set of plasmids encoding truncated TRAIL and a set of plasmids encoding PTEN. In yet another example, the naked DNA plasmids can include a set of plasmids encoding IL-2, a set of plasmids encoding IL-10, and a set of plasmids encoding PTEN or functional fragment thereof. The combination of different anti-tumor mechanisms used against tumor cells has the potential to provide a great advantage in the treatment of solid tumors (e.g., inhibition of benign tumor growth and progression to malignancy) in that multiple mechanisms have the potential to work synergistically.

The novel nucleic acid compositions described herein are specifically designed for localized delivery whether by direct administration to the tumor, the organ containing the tumor, or to the blood or other bodily fluid (e.g., cerebral spinal fluid or lymph) supplying the tumor site or organ containing the tumor. There are several modes available for localized delivery that are described, infra. In general, the novel DNA compositions are formulated simply in saline or buffered solution (e.g., phosphate buffered saline, Tris-EDTA (TE)) without the need for additional facilitating agents or adjuvants.

Nucleic Acid Compositions

The new nucleic acid compositions are useful in inhibiting, controlling, or eradicating tumor growth or progression by inducing or modulating immune responses in an individual. For example, the new nucleic acid compositions can be used therapeutically in malignant tumors. In another example, nucleic acid compositions can be used prophylactically in benign tumors to prevent their growth and progression into malignant tumors. The development of nucleic acid therapeutics has proven to be promising, and the novel DNA compositions can be administered in combination or as a boost with traditional inactivated virus and/or subunit protein or peptide antigen.

The novel nucleic acid compositions (e.g., naked DNA plasmid compositions) have the advantage of containing one or a combination of nucleic acid molecules, some of which encode polypeptides that elicit immune responses and some of which elicit anti-tumor responses (e.g., a combination of constructs encoding IL-2 and constructs encoding IL-10). Additionally, the nucleic acid compositions of the invention are present in a non-viral construct, which is a naked DNA plasmid capable of replicating within a eukaryotic cell and expressing a protein or proteins of interest within that cell. In various embodiments, the nucleic acid compositions described herein do not contain any facilitating agents apart from physiologically compatible solutions (e.g., saline or buffered solution).

DNA sequences (e.g., sequences encoding a cytokine or an anti-tumor protein) can be expressed by operatively linking them to an expression control sequence in an appropriate expression vector and employing that expression vector to transform an appropriate host. Such operative linking of a DNA sequence to an expression control sequence includes, if not already part of the DNA sequence, the provision of an initiation codon, ATG, in the correct reading frame upstream of the DNA sequence.

A wide variety of vector combinations may be employed in expressing cytokines and anti-tumor proteins in tumor cells. Useful expression vectors, for example, can include segments of chromosomal, non-chromosomal, and synthetic DNA sequences. Such vectors may be plasmids, linear DNA, and the like, which will be expressed in a tumor cell. For example, a vector such as pJW4303 (Chapman et al., 1991, Nucleic Acids Res 19:3979-3986; Lu et al., 1996, J Virology 70:3978-3991; Lu et al., 1998, AIDS Res and Hum Retroviruses 14:151-155) can be used. This vector contains approximately 1,600 nucleotides from the cytomegalovirus immediate-early promoter (nucleotides 458 to 2063; GenBank accession number M60231) to drive transcription, and it contains sequences from the bovine growth hormone (nucleotides 2148 to 2325; GenBank accession number M57764) to provide a polyadenylation signal. Vectors may be used with or without heterologous leader peptides, depending on the nature of the sequence to be expressed.

Any of a wide variety of expression control sequences (i.e., sequences that control the expression of a DNA sequence operatively linked to it) can be used in these vectors to express proteins for tumor treatment. Such useful expression control sequences include, for example, the early or late promoters of SV40, CMV, vaccinia virus, polyoma virus or adenovirus, and other sequences known to control the expression of genes of eukaryotic cells or their viruses, and various combinations thereof.

Proper vectors and expression control sequences will include those that are operable in the host (e.g., for expression in mammalian hosts, an appropriate vector will include promoters and other regulatory sequences used for mammalian expression). The vector's copy number, the ability to control that copy number, and the expression of any other proteins encoded by the vector, such as antibiotic markers, will also be considered in selecting a vector.

Other vehicles for expressing DNA in mammalian subjects include live attenuated viral vectors (e.g., recombinant vaccinia (e.g., modified vaccinia Ankara (MVA), IDT Germany), recombinant adenovirus, avian poxvirus (e.g., canarypox (e.g., ALVAC®, Aventis Pasteur) or fowlpox), poliovirus, and alphavirus virion vectors), which have been successful in inducing cell-mediated immune responses. The avian poxviruses are defective in mammalian hosts, but can express inserted heterologous genes under early promoters.

Cytokines

The nucleic acid compositions described herein can encode a combination of two or more cytokines that have been shown to be relevant to tumor immunology (e.g., IL-2, IL-4, IL-5, IL-10, IL-12, IL-15, IL-18, GM-CSF, TNF-α, TGF-β, and IFN-γ). (Mocellin et al., 2001, J Immunother 24:392-407). As seen in Example 5, infra, a naked DNA plasmid composition containing plasmids encoding IL-2 and plasmids encoding IL-10 was successful in increasing longevity in mouse cancer models.

Interleukin-2 (IL-2) is a 17 kD protein (153 amino acids), also known as T-cell growth factor (TCGF). IL-2 is produced by T-cells in response to antigenic or mitogenic stimulation. IL-2 stimulates both natural killer cells and T cells.

Interleukin-10 (IL-10) is a 20 kD protein (178 amino acids) that inhibits the synthesis of a number of cytokines, including IFN-γ, IL-2, IL-3, TNF and GM-CSF produced by activated macrophages and by helper T-cells. IL-10 was shown to have anti-tumor activity in cancer patients by its inhibiting tumor metastasis and growth through the inhibition of tumor angiogenesis (Silvestre et al., 2000, Circ Res, 87:448-52).

The nucleic acid compositions described herein can include a combination of nucleic acid (e.g., DNA) plasmids expressing a cytokine as well as nucleic acid (e.g.,DNA) plasmids expressing a non-cytokine (e.g., anti-tumor proteins).

Nucleic acid compositions encoding other immunomodulatory proteins are also provided. For example, nucleic acid compositions encoding CD80 or CD86 can be used for treating subjects with tumors.

Anti-Tumor Proteins

The nucleic acid compositions described herein include combinations of DNA plasmids encoding cytokines and DNA plasmids encoding non-cytokines (e.g., anti-tumor proteins). The compositions described herein also include DNA plasmids encoding one or a combination of non-cytokine anti-tumor proteins (not in combination with a cytokine(s)). Anti-tumor proteins prevent growth of the tumor through mechanisms inhibiting the growth of cancerous cells. Anti-tumor proteins include those that induce apoptosis, are anti-angiogenic, or allow cell cycle arrest. Examples of anti-tumor proteins include TRAIL, SHP1, SHP2, and PTEN. Full-length versions of these proteins as well as functional fragments of these can be included in the novel nucleic acid compositions.

Tumor necrosis factor Related Apoptosis-Inducing Ligand (TRAIL), also known as Apo-2 ligand or Apo-2L, is a type II transmembrane (TM) protein whose extracellular region forms a soluble molecule upon cleavage. Both membrane-bound and soluble TRAIL proteins induce apoptosis through their death domain-containing receptors and NF-κB activation in many tissues. The selective cytotoxicity of TRAIL to tumor cells, with minimal side effects in animal models, makes TRAIL a candidate as a safe cancer therapeutic. Two variants of TRAIL constructs (TRAIL47 and TRAIL93) in addition to the wild type (wt, full-length) are described herein. These two truncated versions of TRAIL, which start at amino acid 47 or 93, respectively, of the TRAIL protein do not have the TM domain (amino acids 18-38).

Suppressor of High-copy Phosphoprotein phosphatase 1 (SHP 1), also referred to as protein-tyrosine phosphatase SHP-1, is also known as protein-tyrosine phosphatase 1C, PTP-1C, hematopoietic cell protein-tyrosine phosphatase, and SH-PTP1. SHP1 has 595 amino acid residues, and is a 68 kD cytoplasmic protein tyrosine phosphatase that plays a key role in hematopoiesis. This PTPase activity may directly link growth factor receptors and other signaling proteins through protein-tyrosine phosphorylation. The two SH2 regions may interact with other cellular components to modulate SHP1's own phosphatase activity against interacting substrates.

Suppressor of High-copy Phosphoprotein phosphatase 2 (SHP2), also referred to as Protein-tyrosine phosphatase 2C, PTP-2C, is also known as PTP-1D, SH-PTP3, SH-PTP2. SHP2 is a 68 kD cytoplasmic protein tyrosine phosphatase with 593 amino acids residues. This PTPase activity may directly link growth factor receptors and other signaling proteins through protein-tyrosine phosphorylation. It contains two SH2 regions which may interact with other cellular components to modulate its own phosphatase activity against interacting substrates.

Phosphatase and Tensin homolog (PTEN), also referred to as Phosphatidylinositol-3, 4, 5-trisphosphate 3-phosphatase 1, and also called MMAC1 and TEP1 is a tumor suppressor protein which has been demonstrated to possess both protein phosphatase activity and 3′ phosphoinositol phosphatase activity. It has been shown to mediate cellular proliferation and apoptosis. Mutations of PTEN have been found in various human cancers, including glioblastoma, late-stage prostate, breast, lung, and melanoma.

Localized Administration of Nucleic Acid Compositions and Treatment Methods

Localized delivery and absence of facilitating agents can minimize side effects of DNA administration. Additionally, localized delivery can allow a higher concentration of treatment to be administered relative to that allowable by systemic treatment. The invention specifically provides for the delivery of a nucleic acid composition directly to the tumor whether by tumor injection, topical tumor application, mucosal administration, surgical application to the tumor (e.g., benign or malignant), or by administration to a blood vessel supplying the tumor or the organ containing the tumor.

The nucleic acid compositions can be inoculated in or administered to a subject as naked nucleic acid molecules (e.g., naked DNA plasmids) in a physiologically compatible solution such as water, saline, Tris-EDTA (TE) buffer, or in buffered solution (e.g., phosphate buffered saline (PBS)). The compositions described herein can be administered by several modes and routes, all of which are used for localized delivery. They can be administered intratumorally, which can include subcutaneously (SC), intradermally (ID), and/or intramuscularly (IM)(e.g., to treat a sarcoma tumor), and by these routes, they can be administered by needle injection, gene gun, or needleless jet injection (e.g., Biojector™ (Bioject Inc., Portland, Oreg.). Other modes of local administration include oral (e.g., to localize to the stomach or small intestine), intravenous (e.g., via a vessel supplying a specific organ, e.g., the portal vein), intrapulmonary (e.g., via inhalation to localize to regions of the lung), intravitreal (e.g., to localize to an ocular tumor), by administration to cerebral spinal fluid, by administration to lymphoid circulation, and subcutaneous inoculation. Topical inoculation is another possible mode of localized delivery, and can be referred to as mucosal administration. These include intranasal, ocular, oral, vaginal, or rectal topical routes. Delivery by these localized topical routes can be by nose drops, eye drops, inhalants, suppositories, or microspheres, or nanospheres.

Localized delivery is also applicable to solid tumors that are accessible by endoscopy, such as GI system tumors, gastric cancers, and colon cancers. Localized delivery also includes percutaneous injection, such as for tumors of the pancreas, and intraorgan injection such as delivery by endoscopic approach to prostate or to bladder cancers. Localized delivery also includes administration via the blood circulation. For example, administration to the portal vein allows localized delivery to a liver tumor such as a hepatoma; and administration to a blood vessel supplying the lung or a part of the lung allows localized delivery for the treatment of lung cancer. Localized delivery can also include administration of therapy to brain tumors through specially designed pumps such as ALZET osmotic pumps (ALZET Osmotic Pumps, Durect Corporation, Cupertino, Calif.) through cerebral spinal fluid (CSF). Other localized delivery methods can be used as well, such as those developed for traditional chemotherapies.

Another form of localized delivery is by electroporation (e.g., TriGrid Electroporation System, Ichor Medical Systems, San Diego, Calif.). This technology utilizes the application of electrical fields to safely enhance the intracellular uptake of therapeutic agents within a targeted region of tissue. This technique is a physical means to temporarily overcome the impermeable nature of the cell membrane thus enabling a dramatic increase in the uptake of therapeutic agents by targeted cells. Once the cell membrane recovers from the electroporation, it resumes normal function. Thereafter, a therapeutic effect is realized from the activity of the agent transferred into the cell. Since it is a physical response to the application of electrical fields, the electroporation effect can be induced in virtually any type of tissue and has been demonstrated to potentiate the biological effect of a variety of therapeutic agents.

As a general aid to injection into tumors, sonograph or CAT scan visualization of the tumors can be used in conjunction with the injection procedure. In so doing, a dye can be added to the nucleic acid composition to allow for visualizing and monitoring injection of the tumor.

To improve the effectiveness of the nucleic acid composition, multiple injections can be used for therapy or prophylaxis (e.g., as for benign solid tumors with the potential to become malignant) over extended periods of time. To improve immune induction, a prime-boost strategy can be employed. Priming vaccination with DNA and a different modality for boosting (e.g., live viral vector or protein antigen or other traditional vaccine) has been successful in inducing cell-mediated immunity. The timing between priming and boosting varies and is adjusted for each vaccine, e.g., based on a subject's response to the vaccine, the rate of growth and/or regression of the tumor, and other clinical parameters.

The medium in which the nucleic acid composition is introduced should be physiologically acceptable for safety reasons. Suitable pharmaceutical carriers include sterile water, saline, dextrose, glucose, or other buffered solutions (e.g., TE or PBS). Included in the medium can be physiologically acceptable preservatives, stabilizers, diluents, emulsifying agents, pH buffering agents, viscosity enhancing agents, colors, etc.

Two or more immunizations may be necessary, depending on the effect of the immunizations on tumor size. The nucleic acid compositions can be delivered individually or in combination with other DNA therapeutics. A multi-component or multi-antigen formulation can be delivered as a premixed formulation or in multiple separate inoculations. The novel intratumoral DNA compositions can be used alone or in combination with additional modalities of intratumoral vaccines, e.g., live-attenuated, killed, protein or peptide based, viral or bacterial vector based can be delivered. Such a combination can be delivered sequentially (prime+boost) or concurrently in the same or more than one inoculation simultaneously. The novel intratumoral DNA compositions of the invention can be used as either the priming or boosting component or both.

The methods and compositions described herein can be used for treatment of cancers which are characterized by solid tumors. As used herein, the terms “tumor cells” and “cancer cells” are cells having the capacity for autonomous growth, i.e., an abnormal state or condition characterized by rapidly proliferating cell growth. Cancers and solid tumors which can be treated according to methods, and with compositions, described herein, include tumors of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, tumors of head and neck origin, cancer of the small intestine and cancer of the esophagus. Also included are malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary and adenocarcinomas, e.g., carcinomas derived from glandular tissue or in which the tumor cells form recognizable glandular structures. Sarcomas are malignant tumors of mesenchymal derivation.

The nucleic acid compositions described herein can be administered in combination with, or as part of a treatment regimen, that includes other anti-cancer therapies. For example, the compositions may be administered in combination with a second anti-tumor agent. Nonlimiting examples of agents which can be used in combination with the nucleic acid compositions in methods of treating tumors include, e.g., antimicrotubule agents (e.g., paclitaxel, taxotere), topoisomerase inhibitors (e.g., doxorubicin, etoposide), antimetabolites (e.g., 5-fluorouracil (5-FU), methotrexate, 6-mercaptopurine), mitotic inhibitors, alkylating agents, intercalating agents, agents capable of interfering with a signal transduction pathway, agents that promote apoptosis, and antibodies useful for tumor treatment (e.g., antibodies against tumor-associated antigens (including naked antibodies, immunotoxins and radioconjugates). The compositions can also be administered in combination with treatment modalities such as radiation or surgery.

Assessing Treatment with Nucleic Acid Compositions

Efficacy of the novel nucleic acid compositions in treating tumors can be determined by monitoring the size of the treated tumors. A decrease, absence of growth, or reduced rate of growth in the size of the tumors is an indication that an immune response was elicited against the tumor cells. It can also mean that the immune response is working in concert with anti-tumor activity.

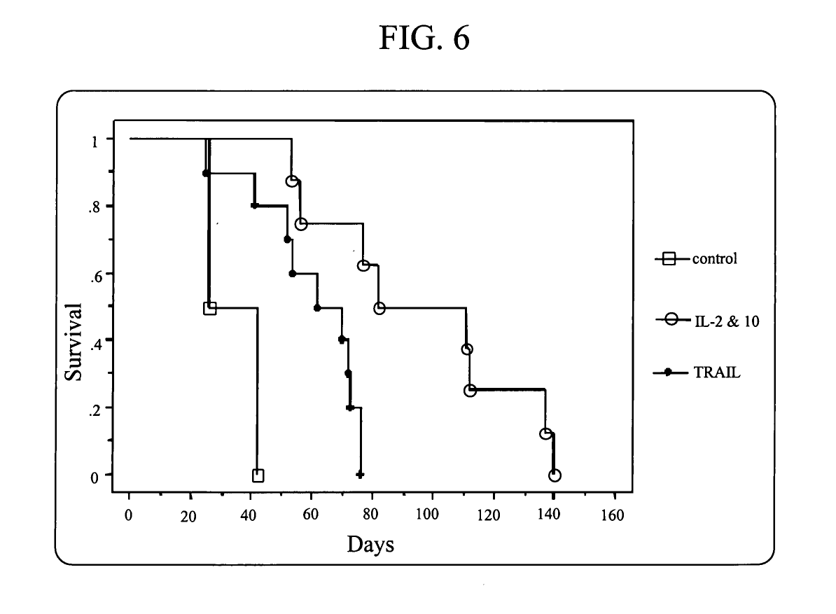

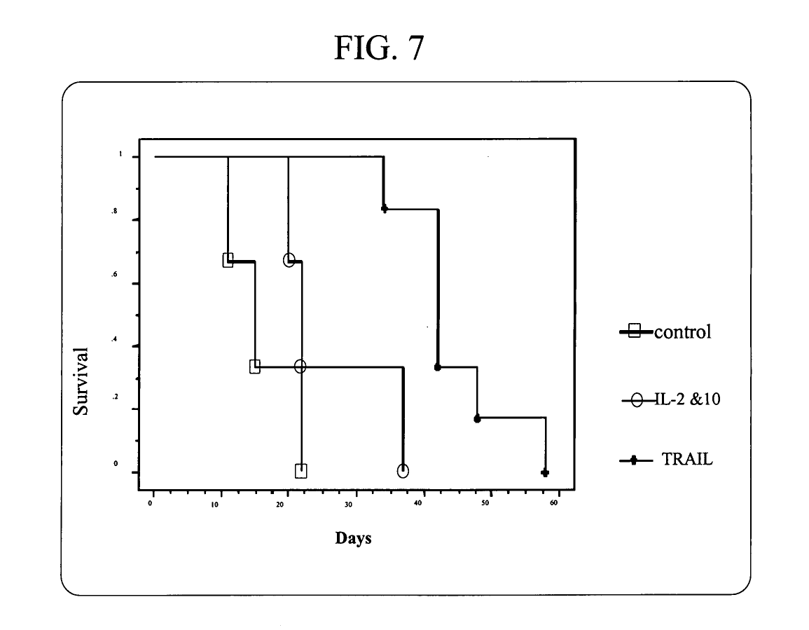

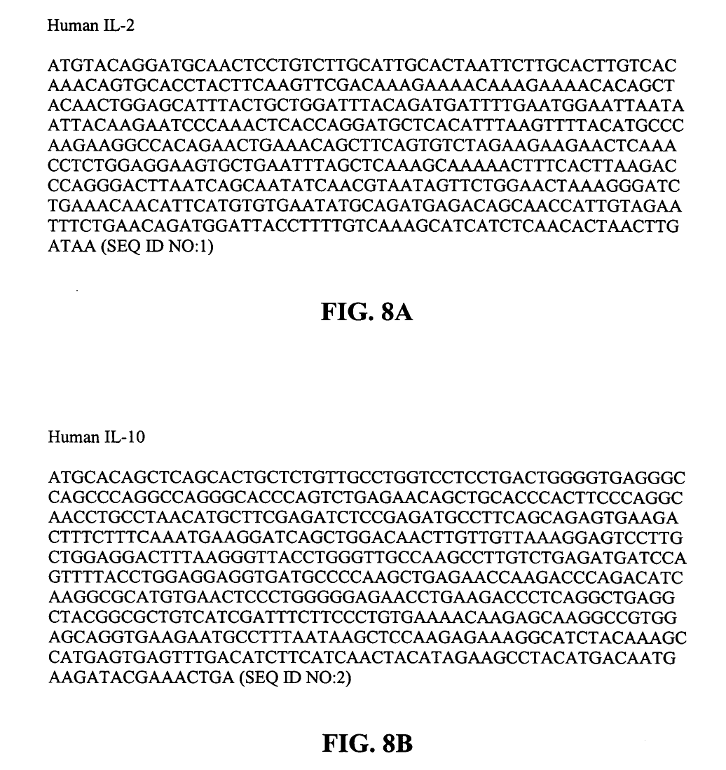

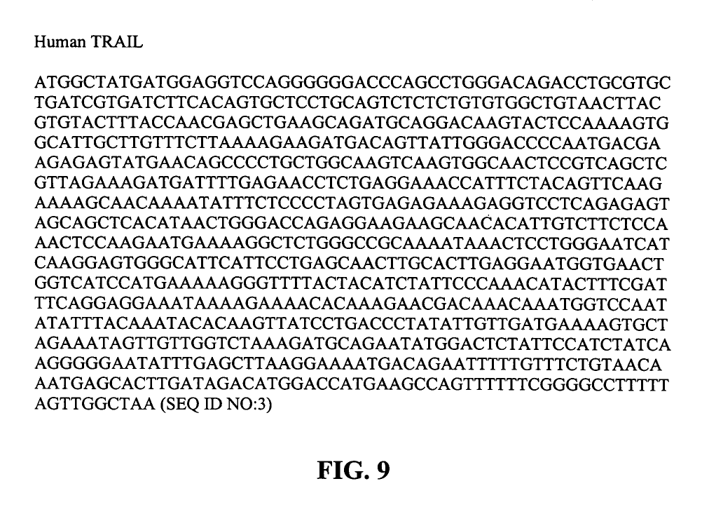

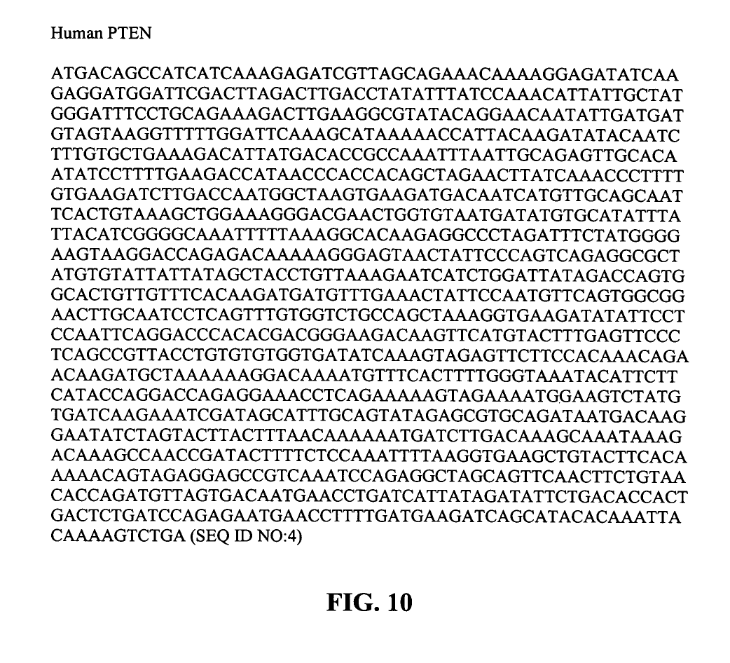

In mouse models of cancer, there can be extensive variability between mice and tumor size relative to survival. Thus, monitoring the survival rate relative to control mice is a preliminary means of testing the novel nucleic acid compositions. Example 4 (FIGS. 6 and 7), infra, provides results in two different mouse cancer models for which the novel nucleic acid compositions were tested and able to increase rate of survival relative to control mice. Further studies can include monitoring tumors within a single subject in which a second tumor, of similar size to the treated tumor, in the same subject, serves as an internal control. In studies in human subjects, these studies can be conducted as described, infra, in Example 5.

Advancements in the field of immunology have allowed more thorough and sensitive evaluation of cellular responses, e.g., to tumors. Such assays as intracellular staining (e.g., flow cytometry) and ELISPOT (an enzyme-linked immunosorbent assay format), allow one to detect and quantify cells producing particular cytokines (e.g., TNF-α and IFN-γ) in response to stimulation with an antigen (e.g., a tumor antigen), thereby allowing one to detect an anti-tumor response. For example, isolation of splenocytes or peripheral blood monocyte cells (PBMCs) from animals or human patients followed by in vitro challenge with an epitope and finally testing by ELISPOT and/or intracellular cytokine staining (ICS), can determine potential for cell-mediated immune response in nucleic acid composition recipients. Flow cytometry using tetramers (i.e., molecules consisting of four copies of a given MHC class I molecule bound to their cognate peptide and alkaline phosphatase) allows the enumeration of antigen-specific T cells (e.g., detection of T cells that recognize specific peptides bound to major histocompatibility complex (MHC) class I molecules). A chromium release assay allows the assessment of cytotoxicity. To assess a cell-mediated immune response to a DNA therapeutic, the more traditional approaches of measuring T cell proliferation in response to an immunomodulating component or antigen and CTL-mediated killing of autologous cells expressing epitopes are also available.

ELISA assays and Western blots can be used to evaluate humoral immune responses, e.g., antibody binding, antibody neutralizing capability, antibody-mediated fusion inhibition, and antibody-dependent cytotoxicity.

### EXAMPLES

The invention is further described in the following examples, which do not limit the scope of the invention described in the claims.

### Example 1

In Vitro Expression of Naked DNA Plasmids

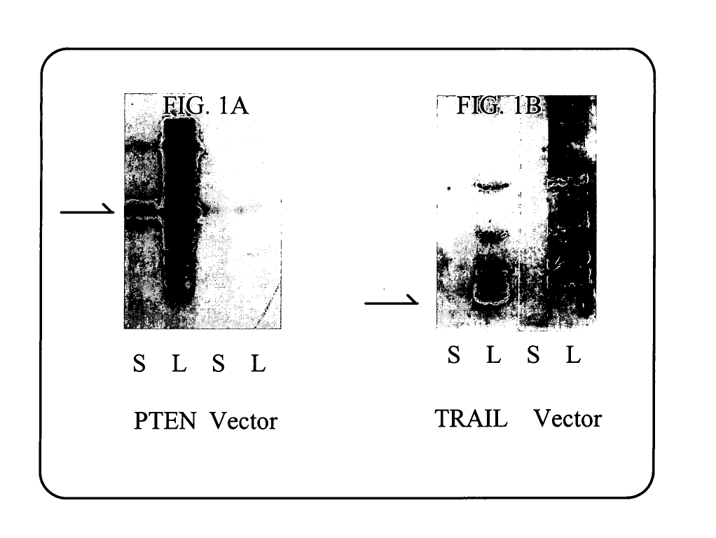

A critical step to verify the quality of gene-based anti-tumor therapeutics is to confirm the expression of coded proteins by naked DNA plasmids by an in vitro assay system. 293T cells were transfected with the pJWS4303 DNA plasmids (10 g DNA per 2 million cells for 32 hours prior to harvesting the cells) using the Ca²⁺-PO₄ method of cell transfection. Cell supernatants and cell lysates were then analyzed by the Western Blot to visualize expressed protein. FIGS. 1A and 1B show expression in 293T cells of a PTEN-containing construct (FIG. 1A) and a TRAIL containing construct (FIG. 1B). PTEN is seen expressed in both supernatant (S) and lysate (L) of the harvested and lysed transfected 293T cells as shown by arrow (FIG. 1A). Wild-type full-length TRAIL was detected in the lysate of 293T cells but not the supernatant (FIG. 1B).

It is known that biological molecules have difficulty entering tumor cells due to changes on the surface of tumor cells. In this study, naked DNA constructs were able to enter tumor cells as shown by expression of the expected protein.

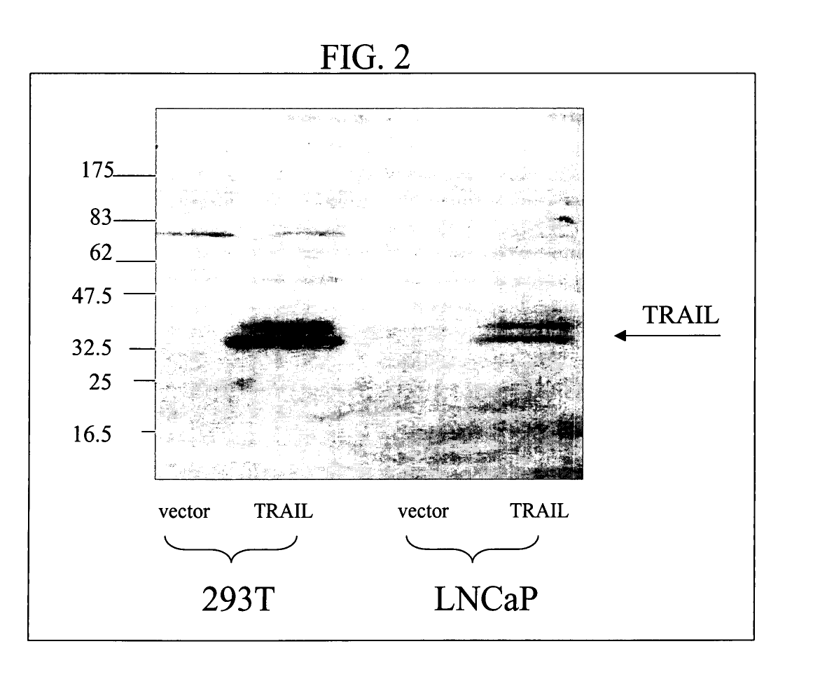

Cells of the human prostate cancer cell line LNCaP were transfected with control and TRAIL-expressing plasmids. These cells were able to express TRAIL protein at easily detectable levels (FIG. 2). Expression of TRAIL in 293T cells was used as a control. Protein expression was determined by standard methods. Briefly, proteins from cell supernatants were separated by polyacrylamide gel electrophoresis. Separated proteins were then transferred to nitrocellulose. Blotting with an antibody recognizing TRAIL followed by secondary antibody and chemiluminescence allowed the detection of TRAIL protein. “Easily detectable” refers to a an amount which is detectable in a short time (e.g., less than one minute) of exposure of an autorad to the chemiluminescent gel such that signal to noise is still minimized and the protein band of interest is visible.

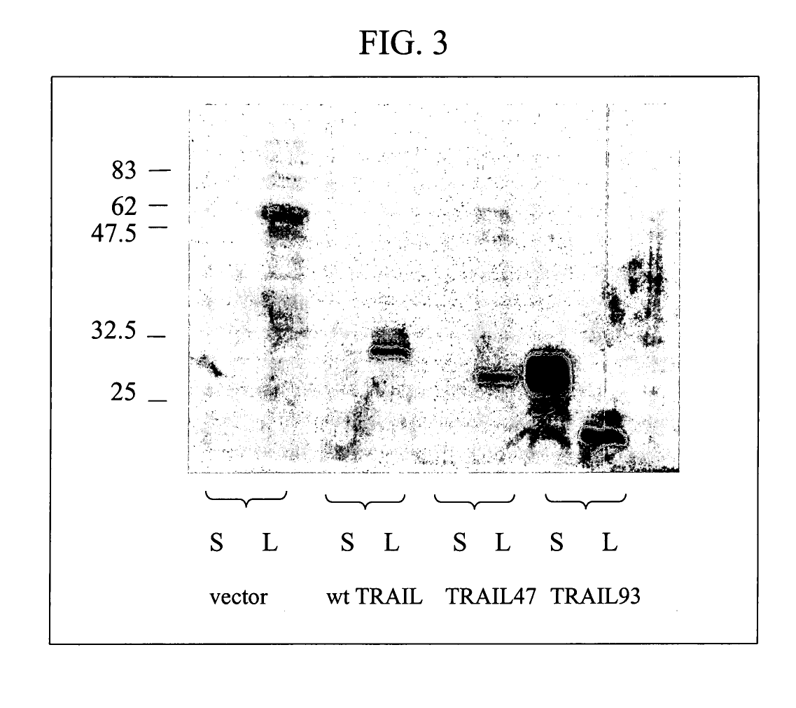

The novel DNA-based anti-tumor approach offers another unique advantage in that the gene insert of a particular anti-tumor protein can be modified to improve its therapeutic effect. Sometimes, such a modification improves intracellular processing of such proteins. Wild-type TRAIL is a type-II transmembrane protein which has unusual intracellular processing post-translation. Three versions of TRAIL inserts were produced: wt TRAIL (amino acids 1-291; amino acid numbering corresponds to the murine sequence), TRAIL47 (amino acids 47-291) and TRAIL93 (amino acids 93-291). FIG. 3 is a picture of a western blot showing expression from constructs with different variants of TRAIL in transfected 293T cells (S: supernatant, L: cell lysate). FIG. 3 shows that the shortest form of TRAIL, TRAIL93, was expressed at much higher levels than the other forms of TRAIL, and a large quantity was secreted. These results indicate that, an in vivo treatment scenario, TRAIL93 can be delivered into targeted tumor tissues in higher doses that TRAIL.

### Example 2

Cell-Based Functional Assays

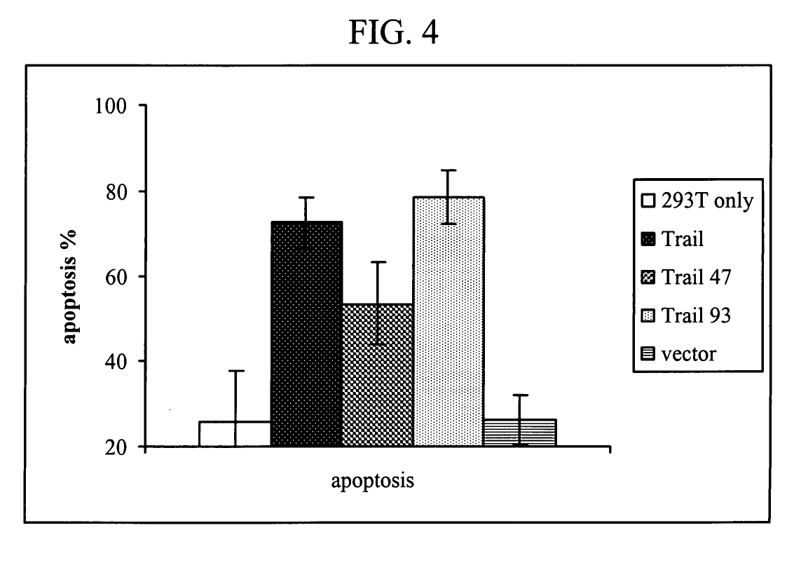

The ability of TRAIL-expressing naked DNA constructs to induce apoptosis of tumor cells in vitro was tested in a cell-based assay. Apoptosis can be measured by many different means known in the art. Here, annexin-V staining was performed to detect apoptosis using a kit by Oncogene (La Jolla, Calif.). FIG. 4 shows that the wild type full-length TRAIL, the truncated TRAIL 47, and the truncated TRAIL 93 were all able to induce significant levels of apoptosis. The shortest version (TRAIL 93) induced apoptosis in cells at the greatest level of the three constructs, with approximately 80% of cells undergoing apoptosis. This is consistent with the in vitro expression result that the TRAIL 93 had the highest expression of TRAIL among these three different designs. Approximately 55% of cells transfected with TRAIL47 were apoptotic. Approximately 75% of cells transfected with wild-type TRAIL underwent apoptosis. These results indicate that nucleic acids encoding wild-type TRAIL and truncated forms of TRAIL lacking the transmembrane domain can be useful for inducing anti-tumor effects.

### Example 3

Intratumor Expression of Gene-Based Anti-Tumor Compositions In Vivo

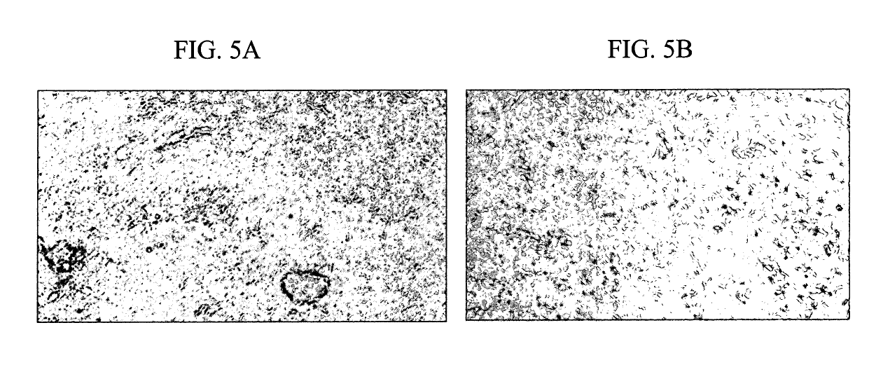

TRAIL-encoding plasmids were inoculated into tumors of mice bearing head and neck tumor cells of the tumor cell line CG200. FIGS. 5A and B shows that expression of TRAIL can be observed in tumor tissue obtained by biopsy from the mice. TRAIL was detected by immuno-histochemical probing with rabbit anti-TRAIL sera (darker regions are positive for TRAIL expression; FIG. 5B). FIG. 5A is a representation tumor cells inoculated with the empty pJW4303 vector as a negative control. This demonstrates that TRAIL can be expressed by intratumoral injection of naked DNA plasmids encoding TRAIL.

### Example 4

Survival Study in Mice

Animal studies were conducted in which mice bearing specific forms of cancer were treated with a nucleic acid composition by direct intratumoral injection. SCID mice were implanted with human prostate cancer LNCaP cells and tumors were allowed to grow to a specified size in each animal prior to DNA treatment (approximately 2-4 months). A total of 100 μg of DNA was administered to each mouse twice a week for four weeks. Mice were injected with plasmids encoding IL-2 and IL-10, TRAIL, or empty plasmids. FIG. 6 depicts survival curves for the mice. Mice treated with plasmids encoding IL-2+IL-10 (n=9) had the longest survival. The median survival time for mice injected with these plasmids was approximately 80 days, with the longest surviving mouse alive for 140 days after tumor implantation. Mice treated with plasmids encoding TRAIL (n=10) also extended the survival relative to those treated with the empty plasmid (pJW4303 vector) (n=4). The median survival time for TRAIL-treated mice was approximately 60 days, with the longest surviving mouse alive for approximately 78 days after tumor implantation. The median survival time for control mice was approximately 20 days, with the longest surviving mouse surviving for approximately 40 days after initiation of treatment. The data indicate that locally administered naked plasmid anti-tumor DNA constructs in saline administered without adjuvant or a facilitating agent were able to extend the life of treated animals.

The DNA compositions were tested in a second tumor model. C57/B6 mice were injected with a head and neck tumor cell line, CG200. CG200 is a spontaneously-arising head and neck tumor cell line derived from a transgenic mouse. Tumors were allowed to grow in mice to a specified size prior to treatment. Naked DNA plasmids containing genes encoding TRAIL, IL-2+IL-10, or empty plasmid were administered twice a week by direct intratumoral injection, at 100 μg total DNA per dose for four weeks. FIG. 7 depicts survival curves of these mice. In this model, the animals treated with plasmids encoding TRAIL (n=8) showed the longest survival. The median survival time was approximately 45 days, with the longest-surviving mouse alive for approximately 57 days after treatment. The median survival time of mice injected with plasmids encoding IL-2 and IL-10 (n=4) was approximately 22 days, with the longest surviving mouse alive until day 40 after tumor implantation. Mice treated with empty plasmid (pJW4303 vector) (n=4) survived for a mediate of approximately 12 days, with the longest surviving mouse alive until day 22. In summary, these data show that intratumorally-administered naked DNA plasmids encoding cytokines and anti-tumor proteins can increase survival times in vivo.

### Example 5

Clinical Trial in Human Subjects

A clinical study in human subjects is conducted to: 1) determine in patients with metastatic malignancies the safety, side effects, toxicity and maximum tolerated dose (MTD) of direct intratumoral injection of increasing doses of a nucleic acid composition designed to introduce the IL-2 gene and the IL-10 gene simultaneously into patients with solid tumors or metastases or lymphomas, 2) confirm in vivo expression of the IL-2 gene and the IL-10 gene in the tumor cells, 3) determine the biological activity and pharmacokinetics of the treatment including: intratumoral gene transfection and transcription by PCR analysis, intratumoral inflammatory or immune response by immunohistochemistry of tumor biopsies, and systemic immune activation as measured by baseline peripheral blood mononuclear cell (PBMC) thymidine incorporation, NK cell activity, and CTL activity against autologous tumor cells, and 4) characterize the clinical response to the study drug by serially assessing the size of the injected tumor and of other tumor masses that may be present and evaluable.

Solid tumors of soft tissue (bony tumors are excluded), and metastases of malignant melanoma, renal cell carcinoma, and hepatic metastases of advanced colorectal carcinoma, and lymphomas are examples of the tumor types that can be evaluated.

Primary tumor nodules are injected several times at intervals of every four weeks with a specified dose of the study drug (see below). There are four groups with five patients each, treated at the prescribed dose (10, 30, 100 or 300 μg), with a group of five patients retreated at the maximum tolerated dose (MTD) dose, or at 300 μg if the MTD is not reached. The highest dose that does not yield Grade 3 or higher toxicities is considered the MTD. All toxicities are graded according to the World Health Organization (WHO) Recommendations for Grading of Acute and Sub-Acute Toxic Effects.

Patients are carefully selected based on their past medical history and present status, if they have failed conventional therapy or if conventional therapy is not indicated, and if they meet the following inclusion criteria: 1) histologically confirmed metastasis of malignant disease, 2) patients must have at least one metastatic lesion measurable in two dimensions and at least 1 cm in longest diameter, 3) patients must have had either prior standard therapies for their disease and have become unresponsive to them, or have made the decision that other therapy would not be of any major benefit, 4) patients must be adults, 18 years of age or older, 5) patients must have adequate bone marrow reserve, 6) estimated life expectancy of a least 16 weeks, 7) patients must be able to render signed informed consent, 8) patients must be HIV antibody negative, Hepatitis B antigen negative and IL-2 antibody negative, 9) female patients who have child bearing potential must use an approved method of contraception and test negative for pregnancy (both male and female patients must use contraception during the course of the study), and 10) patients must demonstrate immunocompetence by having a PHA stimulated lymphocyte response in the normal range.

The study drug is supplied as a single sterile vial containing IL-2 plasmid DNA and IL-10 plasmid DNA in saline or buffered saline in an injection vehicle.

The DNA concentration is specified in the following table:

TABLE OF DNA CONCENTRATIONS

| | Plasmid DNA |
|---|---|
| Dose | Concentration |
| to Patient | (mg/Ml) |
| 10 ug | 0.01 |
| 30 ug | 0.03 |
| 100 ug | 0.1 |
| 300 ug | 0.3 |

The study drug is administered and toxicities are monitored. Tumor lesions are selected for treatment if they are accessible to intratumor administration by direct needle injection. These metastatic lesions are located at any accessible site such as skin, nodes, lung, liver, soft tissues etc. The amount of study drug material injected into each tumor is based on the algorithm outline below. The prescribed dose (10, 30, 100 or 300 μg) is thawed and diluted with injection vehicle to the appropriate volume. If necessary, the study drug is injected with the aid of sonographic or CAT scan visualization of the metastasis. Prior to injection, following placement of the needle, gentle aspiration is applied to the syringe to ensure that no material is injected intravenously. After injection of the drug and with the needle still in place, the dead space is flushed with 0.25-0.50 mL of injection vehicle.

| Tumor Diameter (cm) | Volume of Injection (cc) |
|---|---|
| 1.0-1.5 | 1.0 |
| 1.6-2.0 | 2.0 |
| 2.1-3.0 | 3.0 |
| 3.1-X | 4.0 |

Vital signs are measured every 15 minutes at the start of, during, and after the injection for at least 2 hours or until the patient is stable. If the systolic blood pressure drops below 80 mm Hg, the injection is terminated immediately and the patient is closely monitored and treated appropriately until blood pressure is normalized.

Patients are closely monitored for toxicity for 3-4 hours post injection then 24 hours and 7 days after the first and second injections. For injections 3-6, patients are monitored for 3-4 hours post injection then 7 days post injection as long as they have experienced no toxicity during the 4 and 24 hour observation periods following injections 1 and 2.

TABLE OF SCHEDULE
FOR POST-INJECTION MONITORING

| Treatment | 3-4 Hrs | 24 Hrs | 7 Days | 14 Days |
|---|---|---|---|---|
| 1 | X | X | X | — |
| 2 | X | X | X | — |
| 3 | X | — | X | — |
| 4 | X | — | X | — |
| 5 | X | — | X | — |
| 6 | X | — | X | X |

Before each subsequent injection, patients are evaluated for toxicities from the prior injection and injected with the next dose only if no Grade 3 or higher toxicity occurs. A tumor sizing is done at each intramural injection of the nodule. If the tumor shrinks to a point where it can no longer be injected, subsequent doses are administered into another tumor nodule if any are present.

After the 6th injection, patient follow-up includes evaluations with tumor sizings at weeks 8 and 16. After the week 16 visit, patients are evaluated a minimum of every 4 months.

If a patient experiences stable disease or a partial response (see below) at 4-8 weeks after the last injection of their initial course, he/she may receive an additional course of treatment identical to the first course of treatment or the next higher dose. The patient must, however, continue to meet the entry criteria.

Adverse events are monitored, and patients are removed from the study if unacceptable toxicity (Grade III or IV) develops.

Classical pharmacological studies of drug distribution, half time, metabolism, and excretion are not entirely relevant to in vivo gene injection and expression. However, the fate of the plasmid and detection of the gene products (IL-2 and IL-10) are relevant to the development of this agent. In addition, immune activation is important. Therefore, as part of the measurement of the efficacy of this study, successful gene transfer and expression is evaluated by molecular and immunological analyses.

The following parameters are measured to evaluate the tumor transfection and expression of IL-2 and IL-10: 1) the presence of DNA from the IL-2 gene and DNA from the IL-10 gene are assessed by PCR amplification of cells obtained by biopsy of the treated site after the injection of the study drug, 2) immunohistochemical staining of tumor biopsy samples is used to assess immunologic response and soluble IL-2 and IL-10 expression, 3) serum IL-2 and IL-10 levels are measured, pre-treatment and twice post the start of therapy, however, the detection of serum IL-2 and IL-10 levels is not anticipated, 4) PCR analysis of peripheral blood samples is used to test for the presence of plasmid DNAs after the start of treatment and compared to pre-therapy, but detection of the gene in peripheral blood samples is not anticipated, 5) the cellular immune response is evaluated by measuring baseline and post-treatment IL-2 and IL-10 induced activation of PBMC by thymidine uptake assay and NK/LAK response in peripheral blood pre-therapy and post-therapy, and 6) an attempt is made to excise tumor tissue from another site prior to treatment for diagnosis, immunochemistry, cryo-preservation and to evaluate peripheral blood lymphocyte immunological reactions to the tumor before and after treatment.

As an additional part of the evaluation of the efficacy of this study, the clinical response is measured. Standard oncologic criteria are applied to determine whether or not a patient responds to the study drug. All tumor measurements are recorded in centimeters and constitute the longest diameter and the perpendicular diameter at the widest portion of the tumor. The tumor response definitions listed below are used to compare current total tumor size to pre-treatment total tumor size.

There is a complete tumor response upon disappearance of all clinical evidence of active tumor for a minimum of four weeks, and the patient is free of all symptoms of cancer.

There is a partial tumor response upon fifty percent (50%) or greater decrease in the sum of the products of all diameters of measurable lesions. These reductions in tumor size must endure for a minimum of four weeks. No simultaneous increase in the size of any lesion or appearance of new lesions may occur. The appropriate diagnostic tests used to demonstrate the response can be repeated four weeks after initial observation in order to document this duration.

There is stable disease upon less than 50% decrease in the sum of the products of all diameters of measurable lesions, or an increase in the tumor mass less than 25% in the absence of the development of new lesions.

There is progressive disease upon tumor progression as defined if one or more of the following criteria are met: 1) appearance of any new lesions(s), 2) increase in tumor size of ≧25% in the sum of the products of all diameters of measurable lesions, 3) significant clinical deterioration that cannot be attributed to treatment or other medical conditions and is assumed to be related to increased tumor burden, and 4) worsening of tumor-related symptoms deemed clinically significant by physician.

The principles of informed consent described in Food and Drug Administration (FDA) Regulations 21 C.F.R. Part 50 are followed.

Approval is obtained by the study site Institutional Review Board (IRB) for the Clinical Protocol and Informed Consent Document, and agreement is obtained from the IRB to monitor the conduct of the study and review it periodically.

The primary goal of this protocol is to determine the safety, side effects, toxicity, and maximum tolerated dose (MTD) of direct intratumoral injection of increasing doses of the nucleic acid composition containing IL-2 plasmid and IL-10 plasmid in patients with solid tumors or metastases or lymphomas. Five groups with five patients each can be evaluated at potentially four dose levels. The MTD is defined as the highest dose which does not result in Grade 3 or higher toxicity in any of the patients treated at that dose (it is recognized that the MTD may not be reached). Toxicities and side effects at each dose level are tabulated.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. An isolated nucleic acid composition comprising at least a first set of nucleic acid molecules and a second set of nucleic acid molecules, wherein
(a) each nucleic acid molecule of the first set comprises a sequence encoding a first cytokine or functional fragment thereof and each nucleic acid molecule of the second set comprises a sequence encoding a second cytokine or functional fragment thereof,
(b) each nucleic acid molecule of the first set comprises a sequence encoding a first cytokine or functional fragment thereof and each nucleic acid molecule of the second set comprises a sequence encoding a first non-cytokine, anti-tumor protein or functional fragment thereof, or
(c) each nucleic acid molecule of the first set comprises a sequence encoding a first non-cytokine, anti-tumor protein or functional fragment thereof and each nucleic acid molecule of the second set comprises a sequence encoding a second non-cytokine anti-tumor protein or functional fragment thereof.

2. The nucleic acid composition of claim 1, wherein the nucleic acid molecules comprise DNA vectors.

3. The nucleic acid composition of claim 1, wherein the cytokines of (a) or (b) are selected from the group consisting of IL-2, IL-4, IL-5, IL-10, IL-12, IL-15, IL-18, GM-CSF, TGF-β, TNF-α, and IFN-γ.

4. The nucleic acid composition of claim 1, wherein the cytokines of (a) or (b) are human cytokines.

5. The nucleic acid composition of claim 1, wherein the non-cytokine, anti-tumor proteins of (b) or (c) are selected from the group consisting of an apoptotic protein, an anti-angiogenic protein, and a cell cycle arrest protein.

6. The nucleic acid composition of claim 1, wherein the non-cytokine, anti-tumor proteins of (b) or (c) are selected from the group consisting of tumor necrosis factor (TNF)-related apoptosis-inducing ligand (TRAIL), phosphatase and tensin homolog (PTEN), suppressor of high-copy phosphoprotein phosphatase 1 (SHP1), and SHP2.

7. The nucleic acid composition of claim 1, wherein the non-cytokine, anti-tumor proteins of (b) or (c) are human non-cytokine anti-tumor proteins.

8. The nucleic acid composition of claim 1, wherein each nucleic acid molecule of the first set comprises a sequence encoding a first cytokine or functional fragment thereof and each nucleic acid molecule of the second set of the plurality comprises a sequence encoding a second cytokine or functional fragment thereof.

9. The nucleic acid composition of claim 8, wherein each nucleic acid molecule of the first set comprises a sequence encoding IL-2 or a functional fragment thereof.

10. The nucleic acid composition of claim 9, wherein the sequence encoding IL-2 is at least 80% identical to SEQ ID NO:1.

11. The nucleic acid composition of claim 8, wherein each nucleic acid molecule of the first set comprises a sequence encoding IL-10 or a functional fragment thereof.

12. The nucleic acid composition of claim 11, wherein the sequence encoding IL-10 is at least 80% identical to SEQ ID NO:2.

13. The nucleic acid composition of claim 9, wherein each nucleic acid molecule of the second set comprises a sequence encoding IL-10 or a functional fragment thereof.

14. The nucleic acid composition of claim 8, further comprising a third set of nucleic acid molecules, wherein each nucleic acid molecule of the third set comprises a sequence encoding a non-cytokine anti-tumor protein or functional fragment thereof.

15. The nucleic acid composition of claim 14, wherein the non-cytokine, anti-tumor protein is TRAIL or a functional fragment thereof.

16. The nucleic acid composition of claim 15, wherein the sequence encoding TRAIL is at least 80% identical to SEQ ID NO:3.

17. The nucleic acid composition of claim 14, wherein the non-cytokine, anti-tumor protein is PTEN or a functional fragment thereof.

18. The nucleic acid composition of claim 17, wherein the sequence encoding PTEN is at least 80% identical to SEQ ID NO:4.

19. The nucleic acid composition of claim 1, wherein each nucleic acid molecule of the first set comprises a sequence encoding a first cytokine or functional fragment thereof and each nucleic acid molecule of the second set comprises a sequence encoding a first non-cytokine, anti-tumor protein or functional fragment thereof.

20. The nucleic acid composition of claim 19, wherein each nucleic acid molecule of the first set comprises a sequence encoding IL-2 or a functional fragment thereof.

21. The nucleic acid composition of claim 20, wherein the sequence encoding IL-2 is at least 80% identical to SEQ ID NO:1.

22. The nucleic acid composition of claim 19, wherein each molecule of the first set comprises a sequence encoding IL-10 or a functional fragment thereof.

23. The nucleic acid composition of claim 22, wherein the sequence encoding IL-10 is at least 80% identical to SEQ ID NO:2.

24. The nucleic acid composition of claim 19, wherein the non-cytokine, anti-tumor protein is TRAIL or a functional fragment thereof.

25. The nucleic acid composition of claim 24, wherein the sequence encoding TRAIL is at least 80% identical to SEQ ID NO:3.

26. The nucleic acid composition of claim 19, wherein the non-cytokine anti-tumor protein is PTEN or a functional fragment thereof.

27. The nucleic acid composition of claim 26, wherein the sequence encoding PTEN is at least 80% identical to SEQ ID NO:4.

28. The nucleic acid composition of claim 1, wherein each nucleic acid molecule of the first set comprises a sequence encoding a first non-cytokine, anti-tumor protein or functional fragment thereof and each nucleic acid molecule of the second set comprises a sequence encoding a second non-cytokine, anti-tumor protein or functional fragment thereof.

29. The nucleic acid composition of claim 28, wherein the first non-cytokine anti-tumor protein is TRAIL or a functional fragment thereof.

30. The nucleic acid composition of claim 29, wherein the sequence encoding TRAIL is at least 80% identical to SEQ ID NO:3.

31. The nucleic acid composition of claim 30, further comprising a third set of nucleic acid molecules, each nucleic acid molecule of the third set comprising a sequence encoding a cytokine or functional fragment thereof

32. A nucleic acid composition comprising a first set of nucleic acid vectors, each vector of the first set comprising a sequence encoding IL-2 or a functional fragment thereof, a second set of nucleic acid vectors, each vector of the second set comprising a sequence encoding IL-10 or a functional fragment thereof, and a third set of nucleic acid vectors, each vector of the third set comprising a sequence encoding TNF-related apoptosis-inducing ligand (TRAIL) or a functional fragment thereof.

33. A pharmaceutical composition consisting essentially of the nucleic acid composition of claim 1 and a pharmaceutically acceptable carrier.

34. The composition of claim 33, wherein the pharmaceutically acceptable carrier comprises saline or buffered saline.

35. A method of treating a solid tumor in a subject, the method comprising locally administering the composition of claim 33 to the solid tumor of the subject.

36. The method of claim 35, wherein the composition is administered by intratumoral injection.

37. The method of claim 35, wherein the composition is administered by subcutaneous injection, intramuscular injection, endoscopic percutaneous injection, injection into a vessel supplying blood flow to the solid tumor, injection into a vessel supplying blood flow to a tumor-containing organ, inhalation, localized topical administration, mucosal administration, injection into cerebral spinal fluid, by osmotic pump, or by lymphoid injecting into circulation.

38. The method of claim 35, wherein the composition lacks an adjuvant or facilitating agent.

39. The method of claim 35, wherein the tumor is a prostate tumor or a head and neck tumor.

40. The method of claim 35, wherein the composition is administered in combination with a second agent or treatment modality.

41. The method of claim 40, wherein the second agent or treatment modality is a chemotherapeutic agent, radiation, or surgery.

42. The pharmaceutical composition of claim 33, wherein the pharmaceutically acceptable carrier lacks an adjuvant or facilitating agent.


SEQUENCE LISTING (4 sequences)

SEQ ID NO: 1 - DNA, 474 bp, Homo sapiens
atgtacaggatgcaactcctgtcttgcattgcactaattcttgcacttgtcacaaacagtgcacctacttcaagttcgacaaagaaaacaaagaaaacacagctacaactggagcatttactgctggatttacagatgattttgaatggaattaataattacaagaatcccaaactcaccaggatgctcacatttaagttttacatgcccaagaaggccacagaactgaaacagcttcagtgtctagaagaagaactcaaacctctggaggaagtgctgaatttagctcaaagcaaaaactttcacttaagacccagggacttaatcagcaatatcaacgtaatagttctggaactaaagggatctgaaacaacattcatgtgtgaatatgcagatgagacagcaaccattgtagaatttctgaacagatggattaccttttgtcaaagcatcatctcaacactaacttgataa

SEQ ID NO: 2 - DNA, 537 bp, Homo sapiens
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 3 - DNA, 846 bp, Homo sapiens
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaa

SEQ ID NO: 4 - DNA, 1212 bp, Homo sapiens
atgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga